(19) United States
(12) Patent Application Publication
(10) Pub. No.: US 2025/0127569 A1
(43) Pub. Date: Apr. 24, 2025

(54) Gas-Cooled Laser Device with Plume Removal

(71) Applicant: AEROLASE CORPORATION, Tarrytown, NY (US)

(72) Inventor: Pavel V. Efremkin, Sleepy Hollow, NY (US)

(73) Assignee: AEROLASE CORPORATION, Tarrytown, NY (US)

(21) Appl. No.: 18/917,812

(22) Filed: Oct. 16, 2024

Related U.S. Application Data
(60) Provisional application No. 63/544,826, filed on Oct. 19, 2023.

(51) Int. Cl. A61B 18/20; A61B 18/00

(52) U.S. Cl. CPC A61B 18/203; A61B 18/201; A61B 2018/00017; A61B 2018/00452; A61B 2018/00791; A61B 2218/008

(57) ABSTRACT

A gas-cooled laser apparatus comprises a casing, a laser energy generator disposed of the casing for generating a laser beam, a low-pressure zone and a high-pressure zone provided within the casing. The low-pressure zone generates a vacuuming action while producing a first gaseous stream. The high-pressure zone generates a second gaseous stream. The first and second gaseous streams travel within the casing in the opposite directions. One of the gaseous streams utilized to cool the laser generator is at least partially drawn from a skin treated site by the laser beam. One of the streams drawn from the skin treated site is adapted to vacuum any skin debris generated by the interaction between the laser beam and the skin and directs the debris into the casing interior for further filtration and disposal.

### CROSS REFERENCE TO RELATED APPLICATIONS

This application claims the benefit of U.S. Provisional Application Ser. No. 63/544,826 filed by the applicants on Oct. 19, 2023, which is incorporated in its entirety herein by reference.

### FIELD OF THE INVENTION

The invention is directed to apparatus designed to provide safe and efficient filtration of smoke plume and skin particles generated by laser-surgical devices.

### BACKGROUND OF THE INVENTION

Lasers became a widely used instrument for performing therapeutic and surgery treatments on skin or other soft and hard tissues of humans and animals. During such treatment lasers coagulate, vaporize, or burn tissue that result in producing an airborne plume which may consist of benign smoke, skin or other tissue particles, which may contain live particles, viruses or other potentially harmful materials that may represent a risk to patients and/or partitioners if inhaled.

Surgical smoke and aerosol, or plume, is created when energy is imparted to tissue cells during surgery. For example, when a laser beam is delivered to a cell, heat is created. The heat vaporizes the intracellular fluid, which increases the pressure inside the cell and eventually causes the cell membrane to burst. When this happens, a plume of smoke containing mostly water vapor is released into the atmosphere of the operating room or a doctor's office. At the same time, the intense heat creates chars, protein and other organic matter within the cell and causes thermal necrosis in adjacent cells. The charring of cells releases other harmful contaminants, such as carbonized cell fragments and gaseous hydrocarbons. These small particles and gases are potentially hazardous if inhaled. If they are not evacuated, they become airborne and can be inhaled. This has led to the development and implementation of smoke evacuation systems during surgical procedures. A typical prior art smoke evacuator is basically a vacuum pump with one or more filters designed to evacuate surgical smoke and aerosol from the operating site, filter out essentially all the contaminants, and return the filtered air to the operating room.

Research confirms the effectiveness of these filter media in screening out harmful contaminants. To extend their use, filters may be impregnated with an antimicrobial agent, to inhibit the growth and reproduction of microorganisms that become trapped in the filter.

Currently laser practitioners may use a separate device in the form of a smoke evacuator that vacuums and filters laser skin plume into a disposable filter. Effective use of such separately standing medical vacuums requires help of an assistant who holds often required vacuuming hose of the smoke evacuator near the laser treatment site to catch the plume. Published research indicates that for the currently available medical smoke evacuators the efficient catching of the laser plume requires the end of the vacuuming hose to be not more than one half of an inch from the laser skin treatment site. Otherwise, some of the plume is not caught and escapes into the surrounding air and may potentially harm patients and laser practitioners. Further, such plume can spread through a central air conditioning system to other rooms and areas of a medical office. Published research also shows that because laser treatment typically continues for 30 minutes or more, an assistant who holds the vacuuming hose may lose concentration, this often results in the end of the vacuuming hose to deviate from the skin for more than half an inch, that in turn lead to lasered tissue plume to escape the evacuation system and spread over the air (see FIG. 1).

To save expenses, some practitioners use tape to attach the vacuuming device hose to the laser handpiece or use similar arrangements. However, such arrangements are not working efficiently because they are not adapted to catch all the smoke plumes from the laser treatment. It is typically attached to a side of a laser hand piece thus taking more plume from the one side of the laser skin site to be operated on treatment area while other areas are applied less vacuuming force which may lead to plume escaping the vacuuming hose from that direction (see FIG. 2).

In the context of the application, a “plume” refers to the aerosolized particles and gases that are produced when laser energy interacts with tissue. Such plume typically contains a mix of cellular debris, water vapor, and potentially harmful chemicals, depending on the type of skin tissue being treated. One of the essential aspects of the invention is to manage laser plume effectively in medical settings, to protect both the patient and healthcare providers from inhaling these potentially hazardous substances.

Therefore, there is a clear long felt need for optimally designed medical laser instruments integrating a plume vacuuming function adapted to optimally vacuum the majority of produced laser generated tissue smoke plume. There is a further need for laser instruments which provide substantial advantages for patient and practitioner health by eliminating the risk of inhaling air-borne pathogens. Further, there is a clear unsatisfied need for more economical laser treatment apparatus and method which eliminates the need for an assistant in general, and more specifically eliminating the need for the help of an assistant to hold separately standing auxiliary medical devices, such as for example, the vacuum hose for effective plume removal often disposed near the treatment site.

Recently developed gas or air-cooled medical lasers use an air stream to cool laser generator instead of water flow. Such gas or air-cooled laser devices use negative pressure streams to cool laser generator by vacuuming gas or air through the laser generator located in the handpiece. The construction of such laser handheld devices is similar to vacuum hose where the air enters at one end of the laser hand piece and then travels through the handpiece catching and removing the heat generated by the laser hand piece generator. A general idea of using a filter at the receiving distal end of the handpiece to catch the skin debris was disclosed and utilized in the prior art developed and produced by AEROLASE. However, such prior art devices do not provide a specific design solutions at the distal end of the laser handpiece and disposed in the vicinity of the skin treatment area enabling a user to optimally remove the smoke plume and skin debris produced during the laser treatment.

Another important novel aspect of the invention is the design solutions preventing contamination of laser optical elements surfaces by the gas or air stream containing the smoke plume and skin debris. This relates to both outside laser optical surfaces which are, by design, are located near the laser skin site 9/32 to be operated on the treatment site as well as an internal laser generator optical surface that includes for solid state lasers: laser mirrors, laser crystal ends, as well as other laser generator surfaces. Contamination of those services is detrimental and results in reducing the efficiency of laser generation, i.e., laser output and resulting clinical efficacy of the device.

### SUMMARY OF THE INVENTION

One aspect of the invention provides a laser-based arrangement for skin therapeutic applications, wherein the laser energy generator is cooled by air, and gas or air to cool the laser generator is partially taken from the skin site treated by the laser energy, wherein the gas or air intake from the laser treatment site is optimally designed to vacuum all skin debris produced by laser skin interaction into the laser apparatus for further filtering and disposal.

The air intake from the laser treatment site is optimally designed in such a way that vacuumed gas or air stream from the laser treatment site has uniform radian velocity near the treated skin at the circumference with the center located at laser beam axis on the skin, wherein the gas or air-cooled laser is a solid state laser where lasing elements are cooled by air stream.

Another aspect of the invention provides a laser-based arrangement for skin therapeutic applications, wherein the laser energy generator is cooled by gas or air traveling through the handpiece twice. First supplied by an air pump and second vacuumed into the laser hand piece by a low-pressure zone at the distal and of the handpiece. The vacuuming air stream is also optimally designed to vacuum all skin debris produced by laser skin interaction into the laser apparatus for further filtering and disposal.

Still further aspect of the invention provides a gas-cooled laser apparatus for therapeutic skin applications. The apparatus includes a casing, a laser energy generator disposed within an interior of the casing for generating a laser beam, a low-pressure zone and a high-pressure zone within the casing, the low-pressure zone generating a vacuuming action producing a first gaseous stream, the high-pressure zone generating a second gaseous stream, the first and second gaseous streams are traveling within the casing in opposite directions.

One of the gaseous streams used to cool the laser generator is at least partially drawn from a skin treated site by the laser beam. One of the streams drawn from the skin treated site is adapted to vacuum nearly all skin debris generated by the interaction between the laser beam and the skin, said the stream directs the debris into the casing interior for further filtration and disposal.

Still another aspect of the invention provides an apparatus wherein the laser energy generator is cooled twice by said gaseous streams traveling within the casing. The laser energy generator is cooled by the second gaseous stream generated by the high-pressure zone and is also cooled by the first gaseous stream generated by the low-pressure zone. The first vacuuming stream is also adapted to vacuum substantially all skin debris into the laser apparatus for further filtration and disposal.

Further aspect of the invention provides an apparatus wherein a plum removal unit is formed having outer walls designed with inwardly directed extensions provided to induce air from outside of the plume removal unit for further vacuuming and removal with the plume.

Still another aspect of the invention provides an apparatus outer walls of the plum removal unit are formed with outwardly directed extensions to prevent any plume escaping vacuuming in the plume removal unit. Further a plurality of spacing rods extend outwardly from the outer walls, wherein the spacing rods prevent the plume removal from being drawn toward the skin treated site while blocking the vacuuming stream from entering the internal areas of plume removal unit. Still further a distal end of the plume removal unit is formed with a plurality of openings uniformly distributed at an outer edge thereof. Such openings are designed to draw in air evenly from all directions, while also preventing the plume removal unit from being pulled toward the skin treated site.

### BRIEF DESCRIPTION OF THE DRAWINGS

The subject matter regarding the invention is particularly pointed out and distinctly claimed in the application. The invention may best be understood by reference to the following detailed description when read with the accompanying drawings wherein:

FIGS. 1 and 2 depict prior art arrangements in the field of the invention;

FIGS. 3A, 3B and 3C depict an exemplary diagram according to one preferred embodiment of the invention;

FIG. 4 more specifically illustrates a laser generating assembly disposed in the interior of the inner casing;

FIG. 5 illustrates a further embodiment of the invention, wherein a high-pressure zone is created within the interior region of the inner cone element of the unit;

FIG. 6 illustrates the embodiment of the invention with cooling of the laser handpiece/laser generator with negative pressure at the distal end.

FIGS. 7A and 7B illustrate the embodiment with plume removal unit made in the conical form with cylindrical symmetry around the laser beam axis;

FIGS. 8A, 8B and 8C illustrate the embodiment where the unit has openings uniformly located at the edge of the distant end of unit that optimally designed to take gas or air uniformly from all directions and also prevent unit to be sucked to the skin site to be operated;

FIG. 9 illustrates the embodiment with cooling of the laser handpiece/laser generator facilitated by the negative pressure source at the distal end;

FIG. 10 illustrates another embodiment of the invention where plume removal unit 10 comprises of two channels; and

FIGS. 11A, 11B and 12B illustrate another embodiment of the invention where the unit is adapted to work with standard medical vacuum and optimized for the maximum plume removal.

### DETAILED DESCRIPTION OF EMBODIMENTS OF THE INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

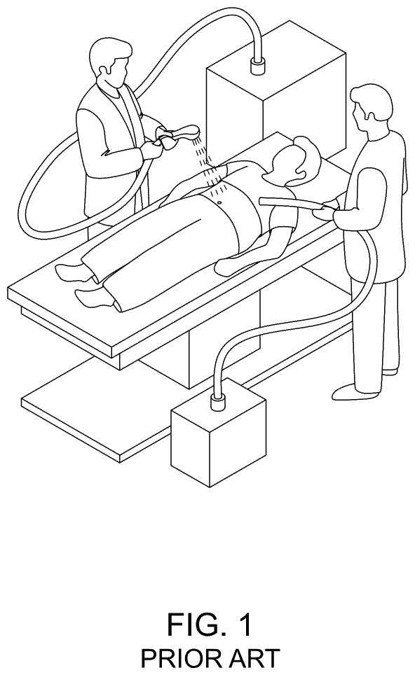

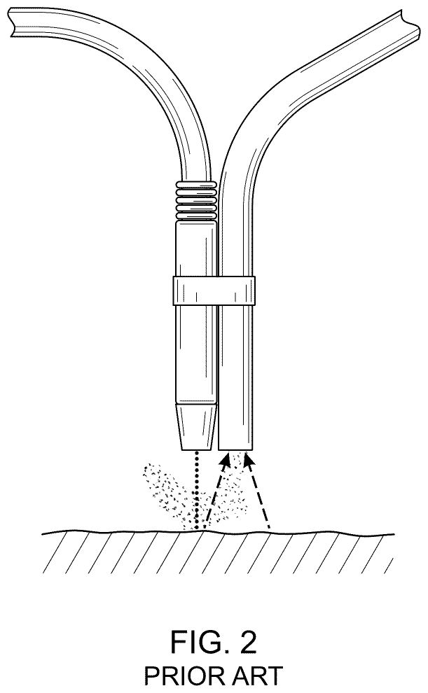

The prior art examples illustrated in FIG. 1 and FIG. 2 are provided to illustrate the use of a laser element and suction holding accessories. There is a laser device and a medical vacuum. In some prior applications it is recommended to tape to attach vacuum hoses to the laser handpiece. As illustrated in FIG. 2, in the prior art when a tape is used to attach the vacuum hose to the laser handpiece, a part of the plume will escape. This is not an efficient way to resolve the situation with the plume evacuation. On the hand, the invention provides the plume removal unit removably connected to the distal end of the laser handpiece and adapted to remove all smoke plumes generated at the skin cite during the laser treatment procedure. And they will create either symmetrical or asymmetrical air streams to remove all the plumes from the area surrounding the optical axis of the laser beam. The air streams containing the smoke plume do uncontrollably penetrate into the internal area of the unit used with the outside vacuum.

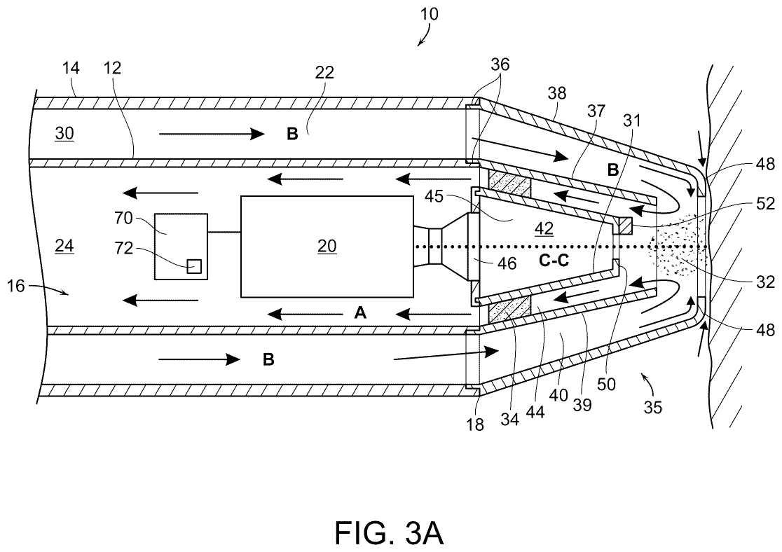

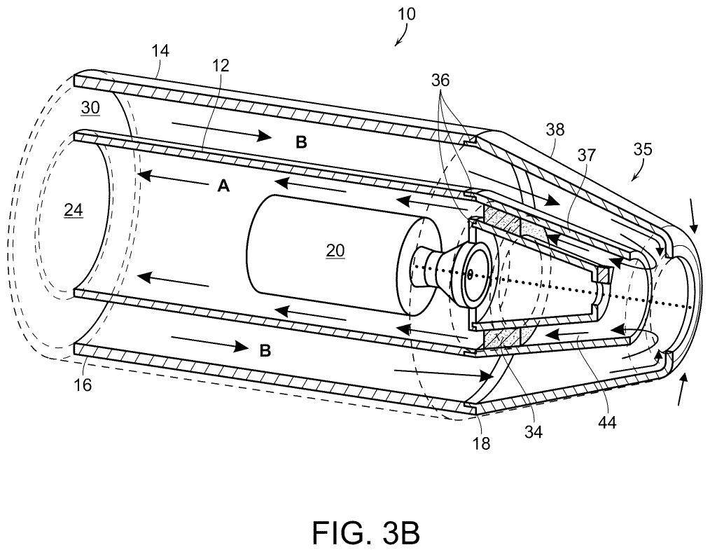

Referring now to FIGS. 3A and 3B, illustrating one embodiment of the invention. The hand-held laser apparatus 10 is formed with an elongated operational inner casing 12 positioned inside the outer casing 14. The inner casing forming a part of a hand piece is formed having a substantially hollow internal area extending between proximal 16 and distal 18 ends. The emitter or a laser generating assembly 20 is provided within an interior of the inner casing. The inner casing is positioned within and surrounded by the outer casing so that these casings are spaced from each other by a hollow space 22. A low pressure or vacuum zone/source 24 is provided at the proximal end 16 of the inner casing. A stream of gaseous coolant can enter the interior space of the inner casing through an inlet opening region disposed at the proximal end 16. In the embodiment of FIGS. 3A and 3B the low-pressure zone or a vacuum 24 provided at the proximal end of the inner casing generates a suction resulted in the gaseous or air stream “A” directed in the distal to proximal ends direction along the laser generator 20 to remove or dissipate heat therefrom. The low-pressure zone 24 can be generated at the proximal end 16 of the inner casing by any conventional means such as an air pump, etc., which can be positioned inside or outside of the inner casing 12. In this manner gaseous coolant or air can be sucked through hand piece or pushed through laser hand piece case (laser generator assembly, laser cavity, laser source, laser generator, any type of laser source: pulsed chip, semiconductor diode, etc.).

In FIGS. 3A and 3B the laser generator 20 can be any solid phase laser regardless of the source of the laser energy.

As further illustrated in FIGS. 3A and 3B a high-pressure zone 30 is also provided at the proximal end 16 of the hand piece which actually directs/pumps the gaseous coolant or air flow “B” into the hollow operational space 22 separating the inner and outer casings. This coolant flow (designated by arrow “B”) passes through operational space 22 in the proximal to distal ends direction so as to ultimately reach the area proximal to the treated skin site 32. At that location the gaseous coolant flow B interacts with the skin of a patient (where the skin particles are produced). At the same time, the intense heat created chars the protein and other organic matter within the cell and causes thermal necrosis in adjacent cells. The charring of cells releases other harmful contaminants, such as carbonized cell fragments and gaseous hydrocarbons and other hazardous by-products of the laser skin tissue interaction.

As also illustrated in FIGS. 3A and 3B, at the skin site 32 the air or gaseous stream B makes 180 degrees turn and is ultimately directed or vacuumed, through the application of the pressure differential produced by the low-pressure zone 24 into the inner or laser generated compartment. In this motion the gaseous stream B passes through the disposable plume removal unit 35 having a filter assembly 34 where it is being filtered/cleaned/purified. The stream B is cleaned and/or filtered as it passes through the inner casing and along the laser generator. Thus, heat produced by the laser generator is removed and the flow is discharged through an exit opening provided at the proximal end of the inner casing of the hand piece.

The conical plume removal unit 35, which can be disposable, is removably connected to the proximal end of the laser handpiece through ports 36 which may incorporate a mechanism preventing re-use of the plume removal unit after a predetermined usage. One of the main functions of the plume removal unit 35 is to remove and dispose of the plume and skin particles collected in filter 34.

As further illustrated in the embodiment of FIGS. 3A and 3B, the plume removal unit 35 extends between front and rear areas. The rear area is connected to the hand piece at the distal end 18. The front area is positioned at the skin site 32 to be operated on when the device of the invention is in use. In the embodiment of FIGS. 3A and 3B the plume removal unit 35 is conically shaped and consists of various areas arranged along the direction of a laser beam axis C-C of the handpiece. More specifically, the plume removal unit 35 is formed by the internal cone 37 positioned inside an external cone 38 having an outer wall 39. The cones are separated by a leading channel 40 providing transition for the gas flow B passing through the operational space 22 to the operation site 32. The internal cone has peripheral or outer wall 39 facing the leading channel 40 and is spaced from a hollow central conduit 42 by the receiving space 44 which is adapted to accommodate the filter 34. When the apparatus of the invention is in the operational mode the low pressure zone is enforced evenly or symmetrically to enter the internal area of the plume removal unit 35 (including the receiving space 44 having the filter 34) in such a way that practically all the plume and skin particles produced by the air laser energy interaction with the skin tissue are drawn or sucked into plume removal unit 35.

In the internal zone 45 of the central hollow conduit 42 the external output laser window or lens 46 are exposed to the gaseous stream contaminated by plume. Without the protection the laser output lens can be seriously damaged causing uncontrollable reduction of the laser energy output. The walls forming the internal cone have a different/sorter length to reduce the internal zone 45. The internal cone is optimally designed so that the air stream “B” coming back or entering the internal area is vacuumed into the receiving space 44 and filter 34. Thus, the plume and skin particles produced at the skin treated area 32 are prevented from entering the internal zone 45 of the central conduit 42 to prevent pollution of the laser lens 46. The design of unit 35 and the ratio between the length of the walls of the external and internal cones walls zone is optimally arranged to have air streams B and A tailored or directed to escape only through the receiving space 44 accommodating the filter 34.

In the plume removal unit 35 the walls of the internal cone 37 are shorter than the walls of the external cone 38 to facilitate vacuuming of the plume into filter 34 and prevent the plume penetration into the internal zone area 45 preventing contamination of the output lens 46.

The outer walls of the unit 35 are formed with the curved or inwardly directed extensions 48 which are optimally designed to induce the air outside of the plume removal unit 35 between the ends of the unit 35 and the skin treatment site for further vacuuming and removal together with the lase generated skin plume. This design produces an even gaseous stream from all directions of the area 32 where the laser beam engages the skin site of a patient to be operated on. An important feature of the internal geometry of the unit 35 is that the air stream does not enter the internal zone 45 accommodating the laser output window and/or laser output focal lens 46 to prevent their contamination by the plume and skin particles. Based on the law of physics extensions 48 of the outer walls are bent inwardly and disposed symmetrically to the optical axis C-C of the laser beam. The air streams within the laser handpiece and unit 35 are optimized to create air movements or streams which also draw the air from outside. In this manner between unit 35 and around the treated skin site 32 the air stream goes into unit 35 internal area, so that practically all plume is being collected and removed through the filter 34. This prevents the escape of the particles of the plume and minimizes/eliminates contamination of air in the treatment room/space which can be harmful to the operator and to the patient.

The curved extensions 48 not only facilitate engagement of the front area of the unit 35 with the skin of the patient at the site 32, but also facilitate the turn of the air stream B containing the smoke plume about 180 degrees form the outward to the inward direction. Another words, the curved extensions 48 facilitate the conversion of the gaseous or air stream direction from travelling along the skin site 32 to the direction toward the interior of the hand piece. Due to the suction generated by the negative pressure source 24 the converted air stream containing the plume and skin particles passes through the receiving space 44 to the filter 34. After filtration the purified air stream is directed to the hand piece interior to cool the laser generator block 20. This arrangement also prevents escape of the hazardous plume and skin particles combination from escaping into the operation/treatment room environment.

The internal peripheral wall 31 has an extension 50 which may be bent inwardly or disposed in parallel to the surface of the output lens 46. On the other hand, the extension 50 can be also positioned substantially paralleled to the plane of skin site 32. All of the above prevent the contaminated air movement to enter into zone 45 of the central hollow conduit. Further, one or multiple censors 52 are provided at the wall extensions 50 to measure concentration of the plume when it approaches the closed/isolated zone 45. Based on the reading of censor 52 the vacuuming source 24 can increase or decrease its vacuuming power to generate suction capable of removing the plume which is produced at the skin site 32 by the laser. This is because the laser can operate at different speeds and levels of energy resulting in variable volume of plume produced at the laser skin or other tissue interaction.

The control unit 70 is provided to regulate the laser energy source/laser generator 20 for the optimum output level and different characteristics of the laser light (such as wavelength, pulse duration, pulse shape, repetition rate etc.) which may be based on type and characteristics of the targeted skin (hard, soft, etc.). Characteristics of control unit 70 may be adjusted either automatically or by an operator. This occurs based on the signals and data received from various sensors such as the sensor 52 etc. provided in the device.

The control unit 70 includes a programmable logic controller or microchip 72 to control the system and apparatus of the invention. The control unit 70 also preferably incorporates control systems for actuating, adjusting and providing system information concerning laser power and other characteristics, which displays reading of the sensors. The control unit 70 may include, but not limited to laser power control unit, vacuum control unit, etc. By means of a computer or microchip 72 the control unit 70 utilizes inputs received from multiple sensors, such as the temperature sensor etc. to continuously update output to an operator including such operating parameters as laser parameters delivered to the skin site, temperature at the skin, and the like.

The control unit 70 is adapted to regulate the laser power source/generator 20 for the optimum output level based on type and characteristics of the targeted skin site (hard, soft, etc.). Characteristics of the control unit 70 may be adjusted by the operator or automatically based on inputs from the sensors. Controlling various characteristics/parameters at the skin perforation site is based on the information provided by the sensors.

Sensors 52, etc. may emit and receive various types of signals (optical, electromagnetic, acoustical, capacitance measuring) that will change parameters depending on the composition of the skin cite, etc., so as to allow the control unit 70 to calculate and generate proper signals controlling operation of the laser generator 20.

Sensors 52, etc. are able to recognize/determine among other data the physical and chemical composition of the patient's skin. The computer or microchip 72 associated with the control unit 70 receives and analyzes information/data obtained by the sensors and generates signals to adjust parameters of the power source, the produced vacuum etc. to optimize the respective medical procedures.

Censors 52 allow optimally regulate the vacuuming power; by increasing the vacuuming power the streams are directed into the receiving space or filter channel. Sensors 52 are adapted to determine physical and chemical composition/characteristics of the plume by means of the computer or microchip 72 of control unit 70 to adjust functionality of the laser. The control unit 70 is provided to generate controlling signals adjusting laser characteristics.

The sensors 52 emit and receive various signals (optical, electromagnetic, acoustical, capacitance measuring) and is capable of detecting composition parameters of the plume, and to allow the control unit 70 to generate controlling signals controlling the skin treatment operation, so that the parameters are detected in the area surrounding the operated skin area by said at least one sensor the control unit 70 generates controlling signals to adjust functionality of the laser.

Although the conical shape of the unit 35 has been illustrated and discussed, it should be obvious to a person of ordinary skill in the present art that various designs of the unit, such as cylindrical for example, are in the scope of the invention.

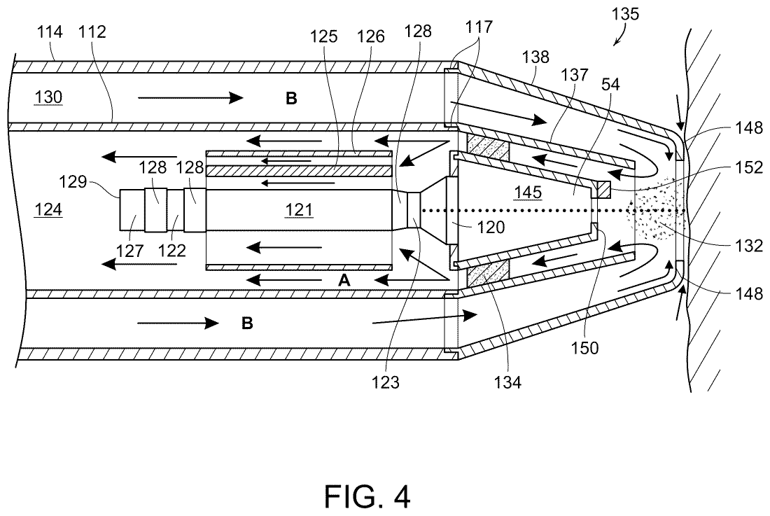

Referring now to the embodiment of FIG. 4 more specifically illustrating another embodiment of the invention having a laser generator 120 disposed in the interior of the inner casing.

The embodiment of FIG. 4 in some respects is similar to the embodiment FIGS. 3A and 3B and discloses the laser-based apparatus adapted for skin therapeutic applications. In FIG. 4 embodiment the laser energy generator is cooled twice by the gas or air stream traveling through the handpiece. The first cooling action is produced by the high-pressure zone which can be in the form of the air pump 130 generating the air or gas stream B moving externally to the laser generating arrangement 120 in the proximal to distal direction. The second cooling action is produced due to the vacuum formed in the laser hand piece by the low-pressure or vacuum source 124 at the proximal end of the handpiece. In this manner the air or gas stream A is produced moving in the distal to proximal ends direction. The vacuum stream A is also optimally adapted to vacuum practically all smoke plume and the skin debris produced by laser skin interaction at the treatment site 132 by being directed into the interior of the laser apparatus for filtering in filter unit 134 and the ultimate disposal.

The embodiment of FIG. 4, instead of a laser generator 20 (see FIGS. 3A and 3B) which can be any solid phase laser regardless of the source of the laser energy, provides a specific design of the solid phase laser having a reflector 126, a flash lamp 125, a laser crystal 121, a laser mirror 122, a laser output mirror 123, and also showing a laser pilot source 127. Specially designed element 128 is an air barrier to protect the space in optical surfaces solid face laser assembly.

The unit 135 (which can be optionally disposable) is detachably connected to the handpiece by means of the connectors 117. Also, the ends of the internal walls of unit 135 may be bent inside or to be parallel to the surface of the output lens to prevent plume entering that internal area of the plume removal unit 135.

As illustrated in FIG. 4 a temperature sensor 129 is provided at the laser generator to regulate the vacuuming power of the vacuum source 124. This occurs through the input of the temperature of the laser generator or emitter to the sensor 129.

Another censor 152 is provided at the wall extensions 150 to measure concentration of the plume when it approaches the closed/isolated zone 145. Based on the reading of censor 152 the vacuuming source 124 is able to increase or decrease its vacuuming power to generate suction capable of removing the plume which is produced at the skin site 132 by the laser.

As a result, the operation of the apparatus of FIG. 4 can be regulated based on two criteria. The first criteria is based on the temperature of the laser emitter inputted by the sensor 129, whereas the second criteria is based on the input of the censor 152 measuring the concentration of the plume when it approaches the closed/isolated zone 145. For the purposes of saving energy the extensive vacuum power should not always be used because the user can use more power than necessary, Thus, utilizing both criteria based on the inputs of sensors 129 and 152 enable the user to apply the optimal vacuuming force, i.e. based on the required temperature of the laser generator and based on the requirement substantially all plume removal. Since this occurs with minimally required energy consumption, the invention provides greener or better ecologically adapted laser-based arrangement for skin therapeutic applications.

To summarize the above, the embodiments illustrated in FIGS. 3A, 3B and 4 disclose a laser-based arrangement for skin therapeutic applications, wherein the laser energy generator is cooled by two air or gas streams traveling through the handpiece in the opposite directions. First air or gas stream A is generated by the low-pressure zone at the proximal end of the handpiece producing the vacuuming action within the laser handpiece. On the other hand, the second air or gas stream B is produced by the air pump generating positive air pressure for cooling the laser generator. The vacuuming air stream A is adapted to vacuum substantially all smoke plume, and the skin debris produced at the interaction of the skin of the patient with the laser beam generated by the laser apparatus by means of passage through filtering and disposal units.

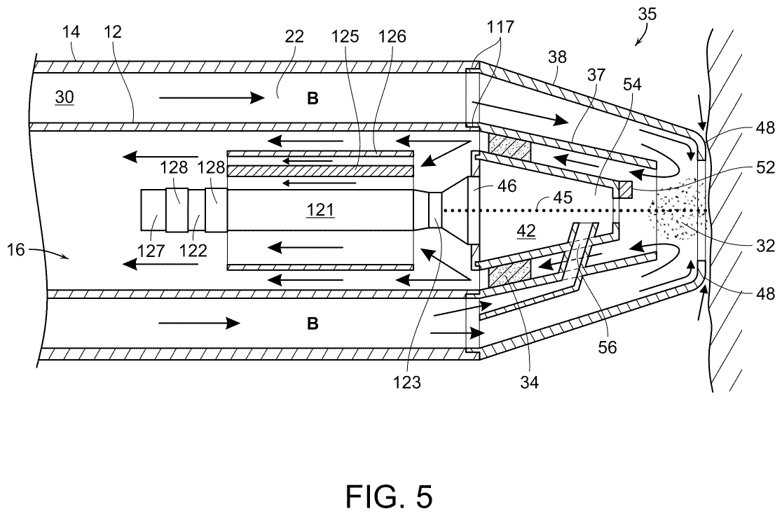

Referring now to FIG. 5 illustrating a further embodiment of the invention. In the internal zone 45 of the central hollow conduit 42 of the inner cone 37 the external output laser window and/or lens 46 can be exposed to the gaseous stream coming from the treatment site 32 contaminated by the plume and skin particles. Without the efficient protection the laser output lens can be seriously damaged/contaminated causing uncontrollable reduction of the laser energy output.

As previously discussed, a high-pressure zone/source 30 provided at the proximal end 16 of the hand piece directs the pressurized gaseous or air flow B into the hollow operational space 22 separating the inner and outer casings. Such air flow B passes through the operational space 22 in the proximal to distal ends direction. The unit 35 (which can be optionally disposable) is detachably connected to the handpiece by means of connectors 117.

The embodiment of FIG. 5 provides an additional protective arrangement within the internal zone 45 of the central hollow conduit 42 formed as an auxiliary high-pressure zone 54 within the internal zone 45 of the central hollow conduit 42 of the inner cone 37. For this purpose, adjustable apertures 56 are formed in the walls of the inner cone elements near the entrance to internal zone 45.

As illustrated in FIG. 5, a portion of incoming pressurized clean air stream B is channeled from the operational space 22 through the adjustable apertures 56 to the internal area 45 forming there an auxiliary high-pressure zone 54.

The pressurized air streams in the auxiliary pressure zone 54 by blowing or pushing air outwardly prevent the contaminated streams produced at the skin site 32 from entering this internal zone 45. In this manner, zone 45 becomes completely sealed from penetration of the smoke plume and skin particles. This is especially important for the protection in internal zone 45 having high-volume lenses which cannot be cleaned easily. As a result, potential contamination of an output lens, laser output mirror and/or optical window of the laser generator also disposed within the zone 45 is also prevented. This further prevents penetration of the air stream contaminated with plume and skin particles generated at the site 32 to the inner space of unit 35.

The air stream B generated by the high-pressure source 30 provided at the proximal end 16 may be pre-cooled by any conventional means of air refrigeration including but not limited to heat pump, chilled water or cryogen spray or liquid. Pre-cooling the air stream B may have an additional benefit of more effectively cooling the laser generator provided in the interior of the handpiece. Further, the pre-cooled air stream B upon being delivered to the skin 32 to be operated on increases comfort to a patient during the laser treatment procedure.

To summarize the above, the proprietary features of the embodiment of FIG. 5 include creation of a of the auxiliary positive pressure area within the internal zone 45 to prevent plume contaminated air passage into the internal area of unit 35 and contaminating/polluting the laser output window/optic of the laser hand piece, wherein the/such positive pressure stream is pre-cooled.

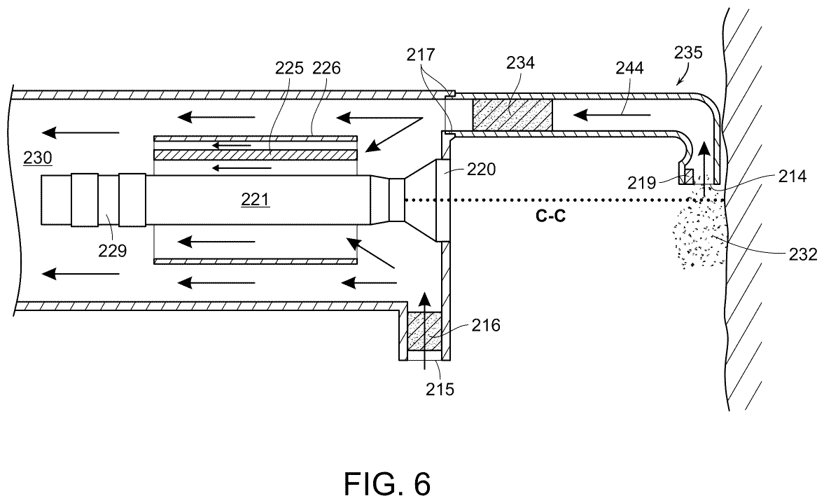

Referring now to FIG. 6 illustrating still another embodiment of the invention, wherein a single source of negative pressure 230 disposed at the proximal end of the handpiece is provided for the plume removal and cooling the laser generator. The smoke plume removal unit 235 is asymmetrically positioned regarding the laser beam axis C-C. The laser handpiece casing apparatus is formed with two input opening formations for air input at the distal end thereof. The function of a first input opening formation 215, 216 is to bring the predetermined amount of air into the interior of the casing provided to effectively cool the laser generator 221. Thus, cooling of the laser generator is facilitated by the air stream entering through the first input opening formation 215, 216 and passing along the laser generator 221 due to the vacuum generated by the single negative pressure source 230.

A second intake opening 214 at the distal part of the unit 235 has a funnel-type configuration. The function of the second input opening 214 is to bring the amount of air necessary to effectively remove the smoke plume and skin debris from the laser treatment site 232 and to direct those to the filter 234. The unit 235 (which can be optionally disposable) is detachably connected to the handpiece by means of the connectors 217.

The unit 235 formed with the receiving space 244 accommodating the filter 234 is asymmetrically positioned with respect to the laser beam C-C. In this manner, the smoke plume and skin particles are drawn into the filter 234 by the negative pressure source 230 from one side of the treatment area 232 only. In view of this geometry, another side of the treatment area/site 232 is not blocked. Thus, the visibility of the treatment site 232 to the operator is not obstructed. It should be noted that in the previously discussed embodiments transparent materials can be used in the manufacturing of the unit 235. However, such material can be contaminated/clouded during the treatment, so that the visibility of treatment site can be substantially reduced. In FIG. 6 embodiment the smoke plume is not only taken from one side of site 232, but also has the ability to remove smoke plume completely. This is facilitated by the funnel-type configuration of the second intake opening 214 of the distal part the unit 235 provided to improve/regulate vacuuming.

When the apparatus of the invention is in the operational mode the low-pressure zone 230 is enforced evenly or symmetrically to enter the internal area of the unit 235 (including the receiving space 244 having the filter 234) in such a way all the smoke plume and skin particles produced by the air laser energy interaction with the skin tissue are drawn/sucked into unit 235. Thus, the plume and skin particles produced at the treatment site 232 are diverted preventing pollution of the laser lens.

The distal part of the unit 235 (especially when it is adapted to contact the skin site 232 to be operated on) may include a sensor 219 to measure plume production temperature at the skin site 232 or any other skin parameters to be used by device of the invention. The previously discussed control unit 70 having the processor 72 used to adjust laser parameters as well as treatment characteristics such as repetition rate, number of laser pulses, etc. based on the signals from the sensors also form a part of this embodiment of the invention.

Further, a temperature sensor 229 can be provided at the laser generator 221 to regulate a vacuuming power the source 230 based on the temperature of the laser generator. The sensor 219 regulates based on the plume production. The apparatus of the invention is regulated based on two criteria: one—based on the temperature of the laser generator, two—based on the ability to remove all the plumes. To save energy consumption it is not necessary to apply an extensive vacuum. Thus, using both sensors 229 and 219 allows applying optimum vacuuming force resulted in the required temperature of the laser generator and all plume removal. However, in the invention this occurs with minimal energy consumed resulted in the greener or better ecologically geared arrangement.

As an optional feature, the distal part of the unit 235 may touch the skin of the patient at the site 232. When the distal part of the unit 235 touches the skin site 232 it can be cooled to provide additional comfort to the patient.

To summarize, in the embodiment of FIG. 6, the air supply streams enter the handpiece in two isolated areas, one at location 214 and another at location 215. This is essential because there are two independent tasks accomplished by the resulting isolated air streams. According to the first task the air stream entering through the opening 215 is used to cool the laser generator 221. This might require grater air volume. Another task accomplished by the air stream entering through the second intake opening 214 of the unit 235 is provided to direct the smoke plume into the filter 234. Having different air intake openings provided in different areas of handpiece enables the user to optimize operation of the device of the invention, so that the opening 215 contributes to optimization by providing an additional air stream to efficiently cool the laser generator. On the other hand, the opening 214 contributes to optimization by removing substantially all smoke plumes produced by the laser. To the best of the inventor's knowledge, the approach has not been disclosed by the prior art, wherein the vacuuming low-pressure air zone generates only one air stream passing the hand piece.

Further, in the embodiment of FIG. 6 the vacuuming air stream consists of two independent sub-streams. One is originated at the distal end of the laser handpiece and the second sub-stream enters the interior of the handpiece through the unit 235 that is optimally designed to vacuum substantially all smoke plume produced by the interaction of the laser beam with skin of the patient. The vacuuming air stream enters the handpiece interior through the unit 235, which is optimally designed to vacuum practically all plumes produced by laser skin interaction from one side of the laser skin treatment area.

This embodiment of the invention provides a novel concept by allowing the design of a handpiece to be optimized for both: cooling the laser generator 221 and also being capable of effectively removing the smoke plume. These requirements are different. By providing a design for unit 235 including filter 234, the goal is to remove substantially the entire plume. This might require a heavy filter which in some instances might reduce the air stream intensity. This is because filtering particles with 0.3 microns or less a very heavy filter comprising of multiple components might be required. In some instances, a charcoal layer might be needed. So that not enough air to cool the laser generator might be produced. By providing separated air intakes for the above-described purposes such intakes are designed to satisfy both criteria. A filter 216 of a different type can be also provided. Filter 234 is provided to filter the smoke plume which includes smoke or other very fine particles up to 0.3 microns. Filter 216 is provided to protect the internal area of the handpiece from penetration into or contamination by larger size particles.

This is to provide an effective cooling for the laser generator and to provide the efficient plume removal. For example, in one embodiment a handpiece has an air stream opening on the side of the sidepiece at 215. In another embodiment, the unit 235 is provided to generate an air stream from the laser treatment laser area through the front of the laser handpiece. Therefore, different diameters accommodating different filters are provided. In this embodiment there is no filter for the air stream for cooling the laser generator. There is practically no need to provide a filter for this purpose. On the other hand, we must filter air stream directed from the treatment site to remove the plume.

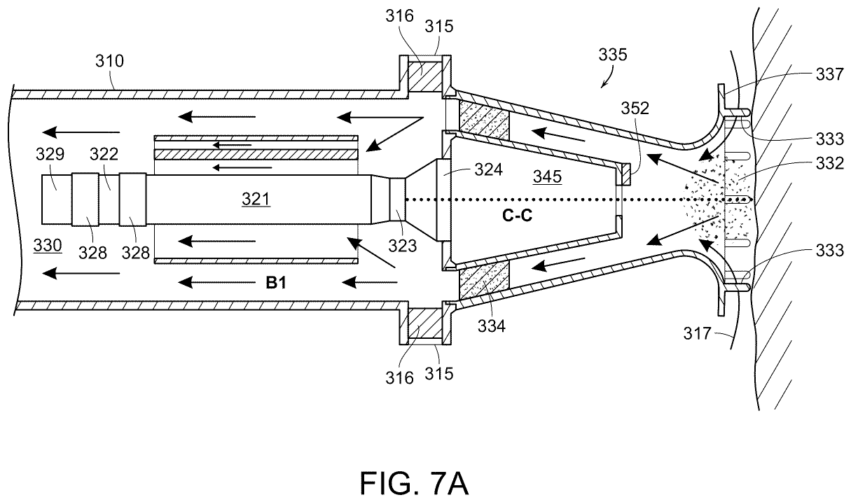

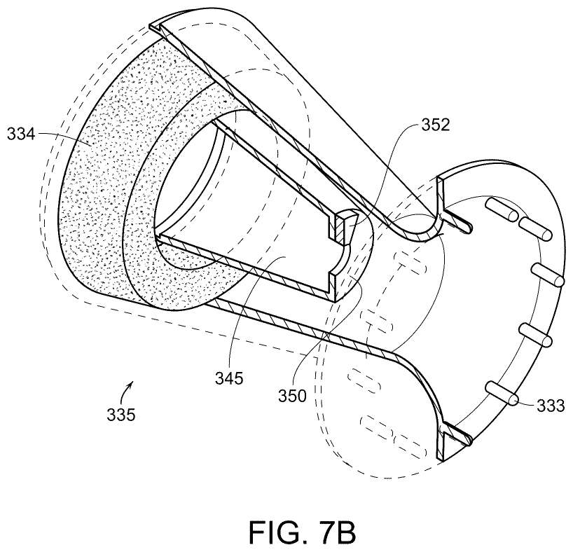

Referring now to FIGS. 7A and 7B illustrating still another embodiment of the invention, wherein the smoke plume removal unit 335 having the conical configuration is symmetrically disposed of the laser beam axis C-C. The distal end 332 of the unit 335 is bent/curved outwardly. Optionally, the distal end 332 of the unit can be parallel to the front surface of laser output lens 324.

The laser handpiece casing 310 of the apparatus of this embodiment is formed with the input opening formation 315, 316 provided at an interface between the distal end of the handpiece and the smoke plume removal unit 335. The function of the input opening formation 315, 316 is to bring the predetermined amount of air into the interior of the casing which is necessary to effectively cool the laser generator 321. Thus, cooling the laser generator is facilitated by the air stream B1 entering through the first input opening formation 315, 316 and passing along the laser generator due to vacuum resulted from operation of the negative pressure source 330 provided at the proximal end.

The second input opening 317 is formed at an interface between the outwardly bent proximal end 332 of the unit 335 and the spacing rods 333. The main function of the second input opening 317 is to bring the amount of air necessary to effectively remove the smoke plume and skin debris from the laser treatment site 332 and direct those to the filter 334.

Multiple censors 352 are provided at the wall extensions 350 to measure concentration of the plume when it approaches the closed/isolated zone 345.

Similar to the previously discussed embodiment of FIG. 6, filter 316 of the different type can be also provided. Previously discussed filter 334 is provided to filter plumes which include smoke and/or other very fine particles up to 0.3 microns in size. The main function of the filter 316 associated with the first input opening formation 315 is to protect the internal area of the handpiece from penetration into and contamination of the handpiece interior by larger size particles.

Spacing rods or members 333 are interposed between the proximal end 337 of unit 335 the skin site 332 to prevent close contact or engagement therebetween. Without the spacing rods 333 or any other spacer the distal end 337 can engage the operational skin site 332. Thus, the suction (vacuum) force can block air coming into unit 335 between the end 337 and the skin site and therefore block the process of the plume removal.

The issue may prevent any air from entering unit 335. This situation is similar to a standard vacuum, which can stop sucking air when positioned against the skin site 332 due to being sealed by the skin or another object. If we generate a vacuum power with unit 335 that exceeds a specific threshold, the end of unit 335, located near the skin site 332 to be operated on, will adhere to the skin. Consequently, this seals the unit, preventing any air from entering. To continue with the laser operation, the laser generator will be overheated because no air stream is coming. To prevent such overheating the special distance (spacing) rods 333 are provided to prevent engagement of the unit 335 to the skin of a patient. The length of the rods 33 should be optimal to prevent the unit 335 from engaging the skin site 332. On the other hand, the length of the rods should prevent the air from escaping from the interior of the handpiece.

To summarize the above, the essential features of the embodiment of FIGS. 7A and 7B include the laser-based arrangement for skin therapeutic applications, wherein the unit 335 has the proximal end 337 bent or curved outwardly to prevent any plume escaping the vacuuming unit 335. Further, the spacing rods 333 interposed between the proximal end 337 of unit 335 and the skin site 332 prevent the unit 335 to engage the skin site, and ultimately prevent vacuuming air stream (containing the smoke plume) to enter the internal area 345 of unit 335.

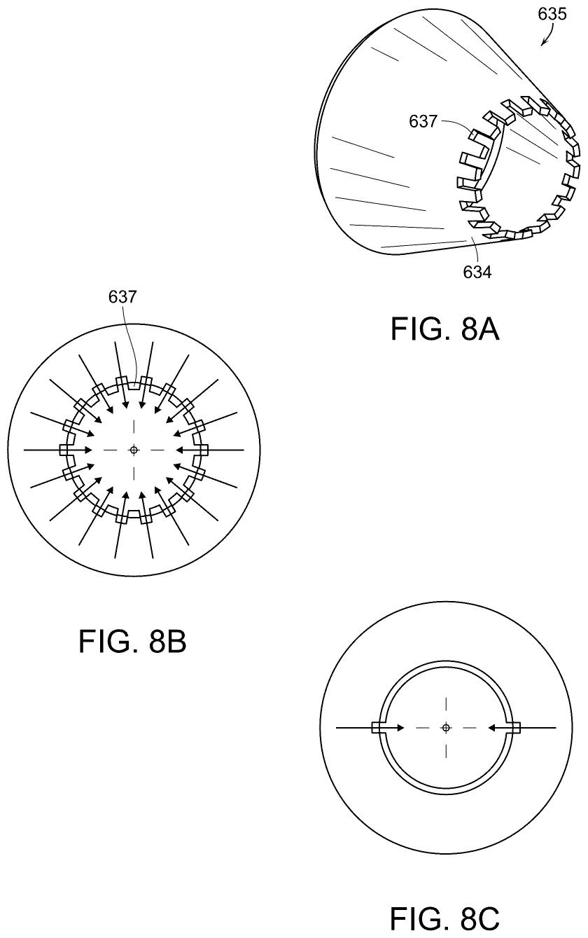

Referring now to FIGS. 8A, 8B and 8C which illustrate still another embodiment of the invention, wherein the unit 635 has openings/slots 637 uniformly located at the edge of the distal end of unit that optimally designed to take air uniformly from all directions and also prevent unit 635 to be sucked and attached to the skin.

The above-noted FIGS illustrate a different embodiment of the spacer rods which can be in the form of cotton which should be uniformed and symmetrical relative to the laser axis.

FIG. 8A shows cuts to prevent distal end 634 to be sucked to the skin site to be operated on by vacuuming force.

FIG. 8B illustrates user axis spot with uniform air streams directed into the smoke evacuation unit.

FIG. 8C Illustrates another embodiment of smoke evacuation plume removal unit with central symmetry. With only two entrances are shown, wherein in FIGS. 8A and 8B there are multiple entrances and elliptical versus circular are provided.

To summarize the above, the proprietary features of the embodiment of FIGS. 8A, 8B and 8C include the unit 635 having the openings/slots 637 uniformly located at the edge of the distal end that optimally designed to take air uniformly from all directions and also prevent unit 635 to be sucked to the skin.

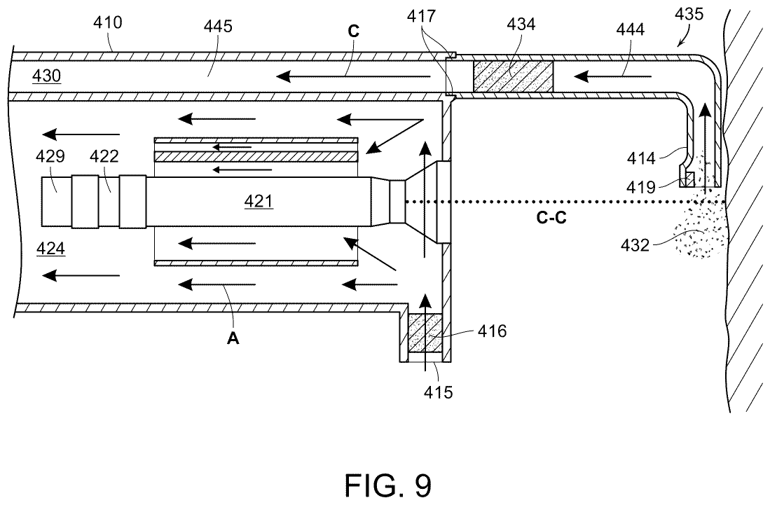

Referring now to FIG. 9 illustrating still another embodiment of the invention having some similarities with the embodiment of FIG. 6. However, in FIG. 9 embodiment there are two independent sources of negative pressure associated with the handpiece. The first independent negative pressure source 424 is provided at the proximal end for the purpose of cooling of the laser generator 421, 422 situated in the handpiece interior. Thus, cooling of the laser generator 421, 422 is facilitated by the air stream A entering the handpiece interior through the first input opening formation 415, 416 to pass along and to cool the laser generator due to the vacuum produced by the first independent negative pressure source 424.

The second independent source of negative pressure 430 also provided at the proximal end of the handpiece for the purpose of vacuuming and removal of the smoke plume and skin particles produced at the skin site 432. The unit 435 formed with receiving space 444 accommodating the filter 434 is asymmetrically positioned with respect to the laser beam C-C. In this manner, the smoke plume and skin particles are drawn into the filter 434 by the air stream produced by the second independent negative pressure source 430 from one side of the treatment site 432 only.

The unit 435 is detachably connected to the casing 410 by means of the connectors 417. The function of the second input opening formation 414 is to bring the amount of air necessary to effectively remove the plume and/or skin debris from the laser treatment site 432 and direct those to the filter 434. In unit 435 the receiving space 444 accommodating the filter 434 is asymmetrically positioned with respect to the laser beam axis C-C. The receiving space 444 is in direct communication with a purified air transition sub-channel 445 (disposed within the handpiece) and associated with to the second independent negative pressure source 430. In this manner the smoke plume and skin particles produced by the air laser energy interaction with the skin tissue are drawn into the filter 434 along with the respective air stream C from one side of the treatment area 432. After passing through the filter 434 the purified air stream is drawn into and transferred through the purified air transition sub-channel 445 to be ultimately disposed.

Provision of two independent sources of negative pressure 424 and 430 enables this embodiment of the invention to carry out two essential functions: (1) to vacuum air from the area surrounding the laser generator for the cooling purposes and (2) to vacuum air including the smoke plum and skin particles from the treatment skin site 432. This may allow a user to regulate and optimize air vacuuming streams for each of the above-mentioned purposes. Having independent air streams to cool the laser generator allows us to optimally regulate the speed of the air stream passing along the laser generator. In the invention this occurs based on the reading of a temperature sensor 429 to optimize the heat removal from the laser generator.

Having the independent air stream C generated by the source 430 to vacuum plume may allow to optimize the speed of vacuuming air stream for more effective plume removal. The latter includes but is not limited to regulating the suction air stream speed based on laser parameters (like energy, repetition rate, etc.), which may increase the volume of plume produced. The optimization process may be also based on reading the sensor 419 that is designed to measure plume concentration at the laser treatment site.

In the embodiment of FIG. 9 there are two independent vacuum streams generated by two independent vacuum sources. One stream generated by the source 424 cools the laser generator, it enters through intake 415, filter 416, and vacuum force is regulated by the temperature of the laser emitter controlled by the temperature sensor 429. Another separate vacuum stream generated by the source 430 is optimized to remove all the smoke plume facilitated by the sensor 419. The temperature sensor 429 is located at the laser generator to regulate vacuuming power or the power of the vacuum source 424 based on the temperature of the laser generator or emitter. The sensor 419 regulates based on the plume production.

The embodiment of FIG. 9 similar to other embodiments of the invention utilizes the computer or microchip 72 associated with the control unit 70 (see FIG. 3A) which receives and analyzes information/data obtained by the sensors and generates signals to adjust parameters such as for example the power source, the produced vacuum etc. to optimize the respective treatment procedures. In this embodiment the treatment process is regulated based on two criteria: one—based on the temperature of the laser and meter, two—the ability to effectively remove all the plumes. This is the optimized solution because it provides vacuuming and air stream to remove all the plume versus another independent vacuuming source and the air stream for cooling the laser generator or emitter.

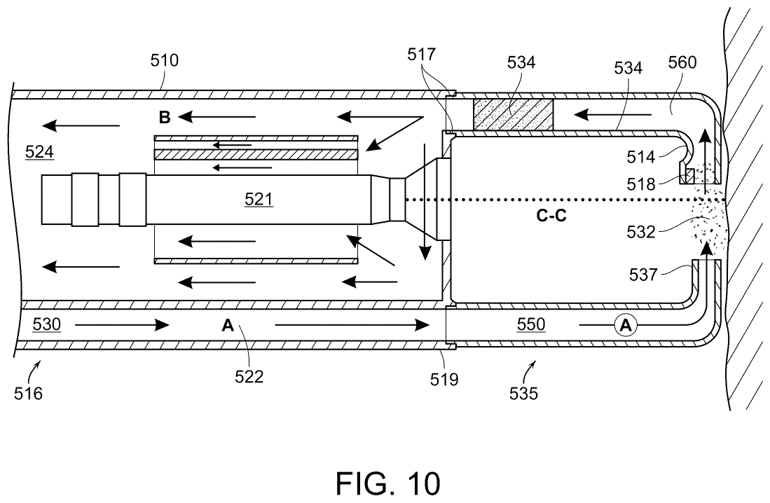

Referring now to another embodiment of the invention illustrated in FIG. 10. The unit 535 is detachably connected to the casing 510 by means of the connectors 517 Similar to the above discussed embodiments a low pressure or vacuum zone 524 is provided at the proximal end 516 of the handpiece inner casing. A high-pressure zone 530 is also provided at the proximal end 516 which directs/pumps the gaseous coolant or air flow into the hollow operational space 522.

As illustrated in FIG. 10, the plume removal unit 535 comprises two channels, symmetrically arranged regarding the laser beam axis C-C, communicating with the distal end 519 of the handpiece. The first channel 550 terminated at a nozzle 537 and connected to the high-pressure zone 530 is used to supply air stream A under positive pressure to the laser skin treatment site 532. The air stream A extends substantially paralleled to the treated skin site 532. Upon passing the treatment site 532 the air stream A catches smoke plumes and skin particles and brings them into a suction zone 545 generated by the negative vacuuming pressure created in the second vacuuming channel 560. Vacuuming/suction is facilitated by the funnel-type configuration of the intake opening 514 of the distal part of unit 535 having wider cross-section than the nozzle 537.

The nozzle 537 and the funnel-type air intake opening or nozzle 514 are optimally configured to organize the air stream near the treatment site to catch all the plumes and move it through intake 514 and channel 544 to the filter 534. The speed of the air stream formed by the opening or nozzle 514 is optimally regulated to catch all the plume and skin particles. This process is enhanced based on readings of sensor 518 provided at the funnel-type air intake opening or nozzle 514 adapted to measure the concentration, speed and other physical characteristics of plume particles.

Another application of FIG. 10 embodiment is based on cooperation of the vacuum air stream and the air stream resulted from operation of the positive air pressure source 530. The pressurized air stream A originated at the source 530 upon approaching the skin site 532 forcibly creates the air stream parallel to the treated area. Then it enters the funnel type intake opening 514 at the other side of the treatment area and passes through the filter 534 to be cleaned and passes the area surrounding the laser generator 521 for the colling purposes. This application provides air stream B which combines the effective removal of the plume and skin particles accompanied by the air-cooling action of the laser generator 521.

There are multiple embodiments disclosed because different types of lasers can be utilized by the apparatus of the invention. There are different amounts of plume, some might have limited amount. It is important to pick up all the plumes and deliver them to the intake. In some situation the plume is minimal and does not require a sophisticated removal arrangement.

To summarize the above, the essential features of the FIG. 10 embodiment include the laser-based arrangement for skin therapeutic applications, wherein, the laser energy generator is cooled by the air stream vacuumed and passing through the laser hand piece and generated by the low-pressure zone at the proximal end of the handpiece. The utilized air stream consists of two sub-streams. One sub-stream is taken from the proximal end of the laser handpiece to cool the laser generator assembly and the second sub-stream separate/independent from the first one is taken through the unit 535 from the vicinity of the laser treatment area to vacuum the plume. The first vacuuming sub-stream is optimized to cool the laser generator and the second sub-stream is optimized for the maximum plume removal. The laser generator cooling is optimized based on reading of the temperature sensor provided at the laser generator. The apparatus is also optimized for the maximum plume removal based on the reading of the plume concentration sensor 518 provided at air intake opening 514 of plume removal unit 535.

Operation of FIG. 10 embodiment can also be regulated by the computer or microchip 72 associated with the control unit 70 (see FIG. 3A) which receives and analyzes information/data obtained by at least the sensor 518 and generates signals to adjust various characteristics of the apparatus to optimize the skin treatment procedures.

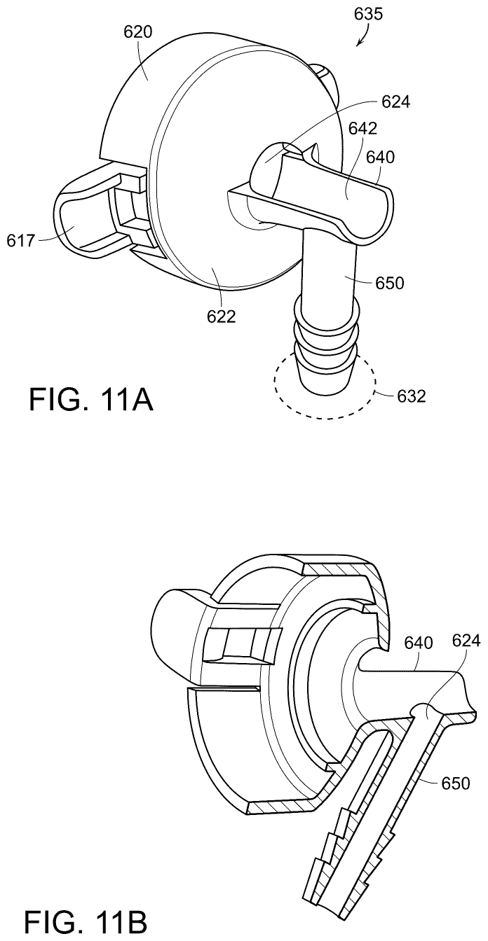

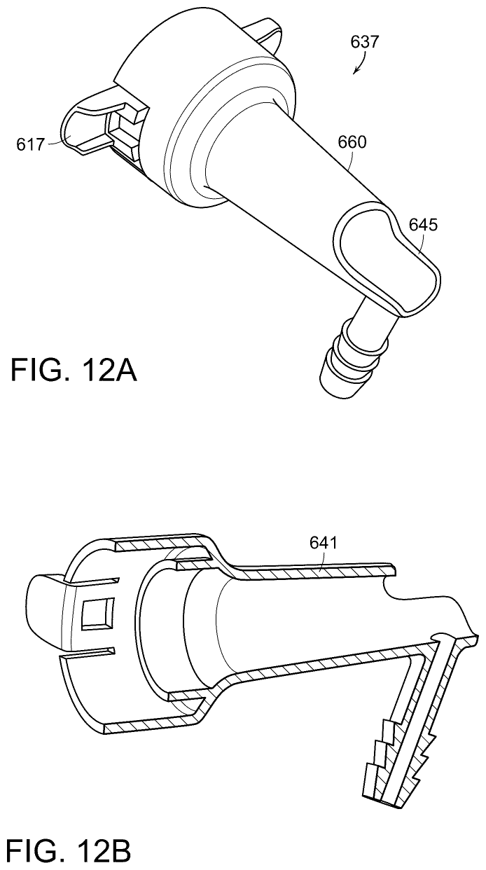

Referring now to further embodiments of the invention illustrated in FIGS. 11A, 11B and 12 A, 12B. Accessary plume removal unit 635 is detachably connected to the distal end of the handpiece by means of connectors 617. As illustrated, the accessory unit 635 is formed by a cylindrical base 620 terminated by a semi-spherical wall 622 having a central aperture 624.

As illustrated in FIGS. 11A and 11B an arm 640 formed as a semi-open tubular wall 642 extends outwardly from the central aperture. The top part of the tubular wall 642 and its connection to the central aperture are not obstructed. A suction pipe 650 extends from a hole 624 provided in the lower portion of the arm. One end of the suction pipe 650 is connected to the hole 644 in the lower portion of the arm and the opposite free end is adapted for positioning in the vicinity of the operated skin site 632 during use of the device. The plume and skin particles are drawn/sucked into the suction pipe by means of the independent vacuum/negative pressure source (typically provided outside of the device).

In use the cylindrical base 620 is movably/rotatably connected to the front/distal end of the handpiece. In this manner, the cylindrical base 620 and the entire accessory unit 635 can be rotatably adjusted about the longitudinal axis of the device or the axis of the laser beam passing through the central aperture 624. In the rotational motion upon finding a predetermined position with respect to the handpiece, the accessory unit 635 is locked at the distal end of the handpiece by means of the connectors 617. As an example, the accessory unit 635 can be rotated from the position illustrated in FIG. 11A to achieve the angular position of the suction pipe illustrated in FIG. 11B.

In view of the illustrated connection between the central aperture 624 and a semi-open tubular wall 640 the visibility of the smoke plume and the skin particles passing from the skin site 632 and through the suction pipe 650 into the tubular wall is not obstructed. Therefore, the operator can observe the stream of plume, etc. passing through the arm into the central aperture 624 and is able to adjust not only the position of the accessory unit 635 at the distal end of the handpiece but also to adjust characteristics of the laser beam intensity.

The diameter of the central aperture 624, the length and configuration of the semi-open tubular wall 642 are selected to accommodate the optimal removal of the smoke plume and skin particles produced at the operated skin site 632. Thus, this embodiment of the invention also accommodates a complete plume removal and prevents contamination of the laser optical lens by the smoke plume and the skin particles.

The embodiment of FIGS. 12A and 12B is similar to the embodiment of FIGS. 11A and 11B. On the other hand, the tubular wall 641 of the arm 660 is modified since only a limited portion at the distal area 645 of the wall is open from the top and unobstructed. This is to assure the visibility of the smoke plume, etc. passing through the arm 660. A substantial part of the wall in the vicinity of connection to the central aperture is closed.

The connectors 617 can be provided with a locking element and are breakable after installation to prevent reuse. During the installation the locking elements lock the accessary 635 at the distal end of the handpiece. The locking elements are adapted to be broken upon removal of the accessory 670 including the locking elements 617 to prevent reuse and contamination for another patient.

In the embodiment of FIGS. 11A, 11B and 12A, 12B the accessories 635 and 637 are provided for the removable connection to the distal end of the handpiece and the standard medical vacuum source 660 generating an air stream that does not touch the laser handpiece output lens is utilized. Thus, these embodiments are applicable for use with the standard laser generating equipment and provide the optimal view of laser treatment site to the operator during the treatment.

While certain features of the invention have been illustrated and described herein, many modifications, substitutions, changes, and equivalents may occur to those skilled in the art. It is, therefore, to be understood that the appended claims are intended to cover all such modifications and changes as fall within the true spirit of the invention.

What is claimed is:

1. A gas-cooled laser apparatus for therapeutic skin applications, comprising:
a casing, a laser energy generator disposed within an interior of the casing for generating a laser beam, a low-pressure zone and a high-pressure zone within the casing, said low-pressure zone generating a vacuuming action producing a first gaseous stream, said high-pressure zone generating a second gaseous stream, said first and second gaseous streams traveling within the casing in opposite directions;
wherein one of the gaseous streams used to cool the laser generator is at least partially drawn from a skin treated site by the laser beam; and
wherein one of the streams drawn from the skin treated site is adapted to vacuum nearly all skin debris generated by the interaction between the laser beam and the skin, said stream directs the debris into the casing interior for further filtration and disposal.

2. The apparatus of claim 1, wherein an air intake from the skin treatment site is optimally arranged to ensure that a vacuuming gaseous stream from the skin treatment site exhibits a uniform radian velocity at the treated skin at the circumference with the center aligned along a laser beam axis.

3. The apparatus of claim 1, wherein said laser generator is a solid-state laser having lasing elements cooled by said gaseous streams.

4. The apparatus of claim 1, wherein the laser energy generator is cooled twice by said gaseous streams traveling within the casing, said laser energy generator is cooled by the second gaseous stream generated by the high-pressure zone and is also cooled by the first gaseous stream generated by the low-pressure zone and said first vacuuming stream is also adapted to vacuum substantially all said skin debris into the laser apparatus for further filtration and disposal.

5. The apparatus of claim 4, further comprising a plume removal unit with an internal area, wherein an arrangement for creating a positive pressure zone is formed within the plume removal unit to prevent plume contaminated gaseous streams from entering into said internal area and to protect optics located within the internal area from contamination.

6. The apparatus of claim 4, wherein said second gaseous stream generated by the high-pressure zone is pre-cooled.

7. The apparatus of claim 4, wherein a plum removal unit having outer walls formed with inwardly directed extensions provided to induce air from outside of the plume removal unit for further vacuuming and removal with the plume.

8. The apparatus of claim 4, wherein said second gaseous stream generated by by the high-pressure zone is directed at one side of the skin treated site to create a stream extending substantially parallel to said treatment site and to capture any plume being carried by the first vacuuming gaseous stream within an area of the plume removal unit located at an opposite side of the skin treated site.

9. A gas-cooled laser apparatus for therapeutic skin applications, comprising:
a handpiece extending between proximal and distal ends, said handpiece having a laser energy generator producing a laser beam, said generator cooled by a vacuuming gaseous stream generated by a low-pressure zone situated at a proximal end of the handpiece; and
wherein said vacuuming air stream consists of two individual streams, with a first individual stream being drawn from the proximal end of the laser handpiece, while a second individual stream is channeled through a plume removal unit adapted to vacuum any plume produced by interaction of the laser beam with a skin treated site.

10. The apparatus of claim 9, wherein outer walls of the plum removal unit is formed with outwardly directed extensions to prevent any plume escaping vacuuming in the plume removal unit.

11. The apparatus of claim 9, wherein the plume removal unit is formed with outer walls, a plurality of spacing rods extending outwardly from the outer walls, said spacing rods preventing the plume from being drawn toward the skin treated site while blocking the vacuuming stream from entering the internal areas of plume removal unit.

12. The apparatus of claim 9, wherein a distal end of the plume removal unit is formed with a plurality of openings uniformly distributed at an outer edge thereof, said openings are configured to draw in air evenly from all directions, while also preventing the plume removal unit from being pulled toward the skin treated site.

13. The apparatus of claim 9, wherein the first individual vacuuming stream is provided to cool the laser generator, while the second individual vacuuming stream is adapted to maximize plume removal;
wherein the laser generator cooling process is optimized based on data provided by a temperature sensor at the laser generator and plume removal process is optimized based on data provided by a plume concentration sensor at the plume removal unit.

14. The apparatus of claim 13, wherein the plume removal unit is adapted for cooperation with a standard medical vacuum equipment, and wherein the plume removal unit is optimized to maximize the plume removal; a vacuuming stream that avoids contact with the laser handpiece output lens is formed; while an operator is provided with an optimal view of the laser treatment site.
